(19) United States
(12) Patent Application Publication
(10) Pub. No.: US 2015/0376723 A1
(43) Pub. Date: Dec. 31, 2015

(54) PROGNOSTIC BIOMARKERS FOR INFLUENZA

(71) Applicant: University Health Network, Toronto (CA)

(72) Inventors: Shaf Keshavjee, Toronto (CA); Marcelo Cypel, Toronto (CA); Shahid Husain, Toronto (CA); Mingyao Liu, North York (CA)

(21) Appl. No.: 14/753,266

(22) Filed: Jun. 29, 2015

Related U.S. Application Data
(60) Provisional application No. 62/018,115, filed on Jun. 27, 2014.

(51) Int. Cl. C12Q 1/70

(52) U.S. Cl. CPC C12Q 1/701; C12Q 2600/158; C12Q 2600/112

(57) ABSTRACT

Described are biomarkers and associated methods for identifying a subject having, or at risk of developing, severe illness from influenza. The level of one or more biomarkers in a test sample from the subject is determined and compared to a control level. Optionally, the subject has or is suspected of having H1N1 influenza. In some embodiments the biomarkers may include IL23R, IL10, TNFRSF13B, CX3CR1, CCR2, MAP2K3 and/or IRF1. In some embodiments, the biomarkers include a combination of IL2 and IL23R or IL10 and IL23R. Also described are methods which include determining the level of two or more biomarkers in a sample and multivariate methods are used for comparing the levels in the test sample to the level in a control sample.

### RELATED APPLICATIONS

This application claims priority to U.S. Provisional Patent Application Ser. No. 62/018,115 filed Jun. 27, 2014, the entire contents of which is hereby incorporated by reference.

### FIELD OF THE INVENTION

The present invention relates to prognostic biomarkers for influenza and more specifically to prognostic biomarkers and associated methods for identifying subjects at risk of developing severe illness from influenza.

### BACKGROUND OF THE INVENTION

The various forms of influenza represent an enormous health burden on the world's population. While some cases of influenza are relatively easy to diagnose and treat, others can progress to more complicated or severe forms of illness that require serious attention or may prove fatal.

H1N1 influenza, commonly known as “swine flu” is a highly contagious respiratory disease generally found in pigs. H1N1 can also be transmitted to humans via contact with infected pigs or contaminated environments and can lead to severe illness.

Accurate predictions of which subjects presenting with symptoms of influenza are more likely to progress to severe illness is expected to improve clinical outcomes by allowing clinicians to make treatment decisions based on a predicted risk of developing severe illness.

Accordingly, there is a need for biomarkers and associated methods for identifying subjects at risk of developing severe illness from influenza.

### SUMMARY OF THE INVENTION

In one aspect, the present invention provides biomarkers and methods useful for identifying subjects having, or at risk of developing severe illness from infection with an influenza virus. In one embodiment, the influenza virus is H1N1, commonly known as “swine flu”. In one embodiment, the biomarkers and methods described herein are useful for identifying subjects having or suspected of having influenza with prior to the onset of severe illness.

As set out in Example 1, samples were taken from subjects presenting with symptoms of influenza and the clinical outcome of each subject was recorded based on whether the subject had no further complications or developed severe illness and required intensive care, required a ventilator, required extracorporeal membrane oxygenation (ECMO), or died. Single and multivariate regression analysis was then used to identify biomarkers and combinations of biomarkers useful for predicting the likelihood of developing severe illness from infection with an influenza virus. In one embodiment, the biomarkers are listed in Table 1. In a preferred embodiment, the biomarkers are listed in Table 2.

The applicants also disclose combinatorial and multivariate approaches for predicting illness severity using the biomarkers disclosed herein. Models based on combinations of biomarkers, optionally 2 biomarkers, 3 biomarkers or more than 3 biomarkers, may be used to identify subjects having or at risk of developing severe illness. In one embodiment, the biomarkers include a combination of two biomarkers listed in Table 3. In one embodiment, the biomarkers include a combination of three biomarkers listed in Table 4.

Accordingly, in one aspect there is provided a method of identifying a subject having, or at risk of developing, severe illness from infection with an influenza virus, the method comprising:
(a) determining a level of one or more biomarkers listed in Table 1 in a test sample from the subject; and
(b) comparing the level of the one or more biomarkers in the test sample to a level of the one of more biomarkers in a control sample, wherein a difference or similarity in the level of the one or more biomarkers in the test sample compared to the control sample indicates that the subject has, or is at risk of developing, severe illness from infection with an influenza virus.

In one embodiment, the subject has or is suspected of having an infection from an influenza virus. In one embodiment, the influenza virus is H1N1 (swine flu).

In one embodiment, the one or more biomarkers or a combinations of biomarkers is selected from Tables 2, 3, 4 or 5. In one embodiment, the one or more biomarkers includes a biomarker selected from IL23R, IL10, TNFRSF13B, CX3CR1, CCR2, MAP2K3 and IRF1.

In one embodiment, the control sample is representative of the level of the one or more biomarkers in subjects presenting with symptoms of influenza whose progression of illness is known.

For example, in one embodiment, the control sample is representative of the level of the one or more biomarkers in subjects with an influenza infection who do not develop severe illness. In one embodiment the marker is IL23R and a lower level of IL23R in the test sample relative to the control sample is indicative of the subject having, or being at risk of developing, severe illness. In one embodiment, the marker is IL10 and a higher level of IL10 in the test sample relative to the control sample is indicative of the subject having, or being at risk of developing, severe illness. In one embodiment, the biomarker marker is TNFRS13B, CX3R1, MAP2K3 or IRF1 and a lower level of TNFRS13B, CX3R1, MAP2K3 or IRF1 in the test sample relative to the control sample is indicative of the subject having, or being at risk of developing, severe illness. In one embodiment, the biomarker is CCR2 and a higher level of CCR2 in the test sample relative to the control sample is indicative of the subject having, or being at risk of developing, severe illness.

In one embodiment, the methods described herein include determining the level of two or more biomarkers in the test sample. In one embodiment, the two or more biomarkers include a combination of two biomarkers listed in Table 3. In one embodiment, the two or more biomarkers include IL23R and IL2. Optionally, the methods described herein include determining the level of IL23R and IL2 and a further biomarker selected from the group consisting of IL15, CXCR6, IRF8, IL10, CD69, CD14, PTGES, KIR3DL1, CD8A and CXCL10. In one embodiment, the two of more biomarkers include IL23R and IL10. In one embodiment, the methods described herein include determining the level of IL23R and IL10 and a further biomarker selected from the group consisting of IL2, MMP3 and IL7R.

In one embodiment, the methods described herein include determining the level of three or more biomarkers in the test sample from the subject. In one embodiment, the three or more biomarkers include a combination of three biomarkers listed in Table 4.

In one embodiment the step of comparing the levels of the one or more biomarkers in the test sample to levels of the one of more biomarkers in a control sample comprises multivariate analysis. Optionally, other methods of statistical or mathematical analysis known to a person of skill in the art can be used to compare the levels of biomarkers in the test sample to the levels of the biomarkers in the control sample.

In one aspect, the level of one or more biomarkers in the control sample is a predetermined or standardized control level. Optionally, the control sample is a sample from the subject at an earlier time point. In one embodiment, a difference or similarity in the level of the one or more biomarkers in the test sample and the control sample over time is used to monitor the subject for progression of illness or response to therapy.

The test sample may be any biological sample that can be tested for the presence or absence of a biomarker described herein. In one embodiment, the test sample is a blood sample, serum sample or plasma sample.

The levels of the biomarkers described herein can be determined in the test samples and/or control samples using methods known in the art. For example, in one embodiment determining the level of one or more biomarkers comprises testing the sample for a nucleic acid molecule or polypeptide encoding for all or part of the biomarker. In one embodiment, testing the sample comprises contacting the sample with a binding agent selective for the biomarker. In one embodiment, testing the sample comprises PCR or RT-PCR.

In another aspect, the methods described herein include treating a subject identified as having, or at risk of developing, severe illness from infection with an influenza virus for severe illness. For example, in one embodiment subjects identified as having, or at risk of developing, severe illness are treated with intensive care. In one embodiment, subjects identified as having, or at risk of developing, severe illness are treated with a ventilator or ECMO.

In another aspect, there is provided a kit for determining whether a subject has, or is at risk of developing, severe illness from infection with an influenza virus. In one embodiment, the kit comprises two or more binding agents selective for two or more of the biomarkers listed in Table 1. In one embodiment, the kit comprises two or more binding agents selective for two or more of the biomarkers listed in Tables 2, 3, 4 or 5. In one embodiment, the two or more binding agents are detectable labeled. In one embodiment, the two or more binding agents are antibodies. Optionally, the kit further comprises a medium suitable for formation of an antigen-antibody complex, reagents for detection of the antigen-antibody complexes and/or instructions for the use thereof. In one embodiment, the kit is configured for performing a method for identifying a subject having, or at risk of developing, severe illness from infection with an influenza virus as described herein.

Other features and advantages of the present invention will become apparent from the following detailed description. It should be understood, however, that the detailed description and the specific examples while indicating preferred embodiments of the invention are given by way of illustration only, since various changes and modifications within the spirit and scope of the invention will become apparent to those skilled in the art from this detailed description.

### BRIEF DESCRIPTION OF THE DRAWINGS

Embodiments of the invention will now be described in relation to the drawings in which:

FIG. 1 shows Area Under the Curve (AUC) values from 10×10 X-Validations for single markers listed in Table 1 where the mean AUC was >65%.

FIG. 2 shows the distribution of markers in 3 marker combinations with AUC≧90%.

FIG. 3 shows the distribution of marker pairs in 3 marker combinations with AUC≧90% (showing pairs occurring>1 combination).

FIG. 4 shows the distribution of marker pairs in the top 15 3-marker combinations with AUC≧90%.

### DETAILED DESCRIPTION OF THE INVENTION

The present description provides biomarkers and combinations of biomarkers that are useful for identifying subjects having, or at risk of developing, severe illness from infection with an influenza virus. In one embodiment, the influenza virus is H1N1 (swine flu). In one embodiment, the subject may present with one or more symptoms of influenza, but does not exhibit symptoms of severe illness.

Accordingly, in one embodiment, there is provided a method of identifying a subject having, or at risk of developing, severe illness from infection with an influenza virus. In one embodiment, the method comprises:
(a) determining the level of one or more biomarkers in a test sample from the subject; and
(b) comparing the level of the one or more biomarkers in the test sample to a level of the one of more biomarkers in a control sample, wherein a difference or similarity in the level of the one or more biomarkers in the test sample compared to the control sample indicates that the subject has, or is at risk of developing, severe illness from infection with the influenza virus.

As used herein a “biomarker” corresponds to a biomolecule such as a nucleic acid, protein or protein fragment present in a biological sample from a subject, wherein the quantity, concentration or activity of the biomarker in the biological sample provides information about whether the subject has, or is at risk of developing, a disease state. In one embodiment the disease state is severe illness from infection with an influenza virus. In one embodiment, the influenza virus is H1N1. In one embodiment, the biomarker is selected from Tables 1-4.

As used herein, “severe illness form infection with an influenza virus” refers to a disease state associated with a viral infection of influenza wherein the preferred clinical treatment includes treatment in intensive care, use of a ventilator or assisted breathing apparatus or ECMO. In one embodiment, signs of severe illness from infection with an H1N1 include one or more of apnea, tachypnea, dyspnea, cyanosis, dehydration, altered mental status, and extreme irritability.

The term “identifying” as used herein refers to a process of determining a subject's likelihood of having, or risk of developing, severe illness from an infection with an influenza virus.

The term “subject” as used herein refers to any member of the animal kingdom. In one embodiment the subject is a mammal, such as a human. Optionally, the subject is a human presenting with one or more symptoms of infection with an influenza virus. In one embodiment, the subject is suspected of having an infection with H1N1, or has been in contact with one or more subjects having or suspected of having an infection with H1N1.

In one embodiment, subjects having, or suspected of having, an infection with H1N1 influenza virus present with symptoms of acute respiratory illness. In one embodiment, subjects having, or suspected of having an infection with H1N1 influenza present with at least 2 of the following: fever, cough, sore throat, body aches, headache, chills and fatigue and diarrhea and vomiting.

In one embodiment, subjects having or suspected of having an H1N1 infection are identified using the CDC criteria as follows:
Onset of acute febrile respiratory illness within 7 days of close contact with a person who has a confirmed case of H1N1 influenza A virus infection, or
Onset of acute febrile respiratory illness within 7 days of travel to a community (within the United States or internationally) where one or more H1N1 influenza A cases have been confirmed, or
Acute febrile respiratory illness in a person who resides in a community where at least one H1N1 influenza case has been confirmed.

In one embodiment, the methods described herein include comparing the level of one or more biomarkers in a test sample to a level of one of more biomarkers in a control sample. The term “sample” as used herein refers to any fluid or other specimen from a subject that can be assayed for biomarker levels, for example, blood, serum, plasma, saliva, cerebrospinal fluid or urine. In one embodiment, the sample is whole blood, blood plasma or serum.

The term “level” as used herein refers to the quantity, concentration, or activity of a biomarker in a sample from a subject. In one embodiment, the biomarker is a protein or protein fragment and the biomarker is detected using methods known in the art for detecting proteins such as ELISA or mass spectroscopy. In one embodiment, the biomarker is a protein or mRNA and the level is an expression level of the corresponding protein or mRNA. Optionally, the biomarker is an enzyme and enzyme activity levels are determined in a test sample from a subject to indicate a level of the biomarker in the subject. Optionally, biomarker mRNA levels or cDNA levels are determined in a test sample from a subject to indicate expression levels of the biomarker in the subject.

As used herein, the term “control sample” refers to a sample representative of one or more subjects whose status and/or clinical outcome following infection with influenza is known. In one embodiment, the control sample is representative of subjects who presented with one or more symptoms of infection with an influenza virus but did not progress to severe illness. In one embodiment, the influenza virus is H1N1.

Optionally, the control sample is age-matched or matched for ethnicity or genetic background with the subject who provides the test sample. In one embodiment, the one or more biomarker levels in the test sample are compared to levels of one or more biomarkers in a control sample. Optionally, the phrase “level of one or more biomarkers in a control sample” refers to a predetermined value or threshold of a biomarker or levels or more than one biomarker, such as a level or levels known to be useful for identifying subjects having, or at risk of developing, severe illness from infection with an influenza virus.

In a further embodiment, the method includes comparing biomarker levels in samples taken from a subject at different time points. For example, in one embodiment, the control sample is determined from a test sample taken from a subject at an earlier time point. Accordingly, the methods described herein may be used to monitor the progression of illness or clinical response to therapy in a subject or group of subjects at different time points.

In one embodiment, the step of comparing the level of one or more biomarkers in a test sample to a level of one of more biomarkers in a control sample can be performed by any one of a number of methods known in the art. For example, in one embodiment the levels of individual biomarkers, such as those listed in Tables 1 or 2, are compared to determine if there is a difference indicative of the subject having, or at risk of developing, severe illness from infection with an influenza virus. As set out in Example 1, differences in the levels of certain biomarkers in a test sample compared to a control sample are indicative of severe illness in subjects presenting with influenza.

In another embodiment, levels from more than one biomarker are compared to identify a subject having, or at risk of developing, severe illness from infection with an influenza virus. Methods that can be used to compare levels in test samples and control samples include, but are not limited to, analysis of variance (ANOVA), multivariate linear or quadratic discriminant analysis, multivariate canonical discriminant analysis, a receiver operator characteristics (ROC) analysis, and/or a statistical plots.

A person skilled in the art will appreciate that a number of different methods are useful to determine the level of the relevant biomarkers of the invention. In one embodiment, the level of the relevant biomarkers of the invention may be determined by real time PCR or other methods known in the art for determining gene expression. In one embodiment, the methods use mass spectroscopy for detecting biomarkers in a sample from a subject. In one embodiment, protocols for determining the level of biomarkers use agents that bind to the biomarker protein of interest. In one embodiment the agents are antibodies or antibody fragments. The term “antibody” as used herein is intended to include monoclonal antibodies, polyclonal antibodies, and chimeric antibodies. The antibody may be from recombinant sources and/or produced in transgenic animals. The term “antibody fragment” as used herein is intended to include Fab, Fab′, F(ab′)2, scFv, dsFv, ds-scFv, dimers, minibodies, diabodies, and multimers thereof and bispecific antibody fragments. Antibodies can be fragmented using conventional techniques. For example, F(ab′)2 fragments can be generated by treating the antibody with pepsin. The resulting F(ab′)2 fragment can be treated to reduce disulfide bridges to produce Fab′ fragments. Papain digestion can lead to the formation of Fab fragments. Fab, Fab′ and F(ab′)2, scFv, dsFv, ds-scFv, dimers, minibodies, diabodies, bispecific antibody fragments and other fragments can also be synthesized by recombinant techniques.

Antibodies having specificity for biomarker proteins may be prepared by conventional methods. A mammal, (e.g. a mouse, hamster, or rabbit) can be immunized with an immunogenic form of the peptide which elicits an antibody response in the mammal. Techniques for conferring immunogenicity on a peptide include conjugation to carriers or other techniques well known in the art. For example, the peptide can be administered in the presence of adjuvant. The progress of immunization can be monitored by detection of antibody titers in plasma or serum. Standard ELISA or other immunoassay procedures can be used with the immunogen as antigen to assess the levels of antibodies. Following immunization, antisera can be obtained and, if desired, polyclonal antibodies isolated from the sera.

To produce monoclonal antibodies, antibody-producing cells (lymphocytes) can be harvested from an immunized animal and fused with myeloma cells by standard somatic cell fusion procedures thus immortalizing these cells and yielding hybridoma cells. Such techniques are well known in the art, (e.g. the hybridoma technique originally developed by Kohler and Milstein (Nature 256:495-497 (1975)) as well as other techniques such as the human B-cell hybridoma technique (Kozbor et al., Immunol.Today 4:72 (1983)), the EBV-hybridoma technique to produce human monoclonal antibodies (Cole et al., Methods Enzymol, 121:140-67 (1986)), and screening of combinatorial antibody libraries (Huse et al., Science 246:1275 (1989)). Hybridoma cells can be screened immunochemically for production of antibodies specifically reactive with the peptide and the monoclonal antibodies can be isolated.

In one embodiment of the invention, the agents, such as antibodies or antibody fragments, that bind to the biomarker of interest such as a biomarker listed in Table 1 are labeled with a detectable marker. The label is preferably capable of producing, either directly or indirectly, a detectable signal. For example, the label may be radio-opaque or a radioisotope, such as ³H, ¹⁴C, ³²P, ³⁵S, ¹²³I, ¹²⁵I or ¹³¹I; a fluorescent (fluorophore) or chemiluminescent (chromophore) compound, such as fluorescein isothiocyanate, rhodamine or luciferin; an enzyme, such as alkaline phosphatase, beta-galactosidase or horseradish peroxidase; an imaging agent; or a metal ion. In another embodiment, the detectable signal is detectable indirectly. For example, a labeled secondary antibody can be used to detect the protein of interest.

A person skilled in the art will appreciate that a number of other methods are useful to determine the levels of biomarkers in a sample, including immunoassays such as Western blots, ELISA, and/or immunoprecipitation followed by SDS-PAGE immunocytochemistry etc. Other embodiments include the use of methods for determining levels of a biomarker in a sample such as lateral flow and related immunochromatic tests used in point-of-care tests. In addition, protein arrays (including microarrays) are useful. For nucleic acid biomarkers such as mRNA, RT-PCR or quantitative RT-PCR or other methods known in the art for detecting and/or quantifying nucleic acids are also useful for determining the level of a biomarker for use in the methods described herein.

The invention also includes kits for identifying subjects at risk of developing severe illness from an infection with an influenza virus comprising a detection agent for biomarkers described herein, typically with instructions for the use thereof. In one embodiment, the kit includes antibodies directed against two or more biomarkers listed in Table 1. In one embodiment, the kit includes antibodies directed against IL23R and IL2. In one embodiment, the kit includes antibodies directed against IL23R and IL10.

In one embodiment, the kit optionally includes a medium suitable for formation of an antigen-antibody complex, reagents for detection of the antigen-antibody complexes and instructions for the use thereof. In an additional embodiment, the invention relates to a composition comprising an, optionally provided together in a container.

The above disclosure generally describes the present invention. A more complete understanding can be obtained by reference to the following specific examples of certain embodiments of the invention.

### Example 1

### Identification of Prognostic Biomarkers for Severe Illness in Subjects Presenting with H1N1 Influenza

### SUMMARY

Serum samples were collected from solid organ transplant recipients (SOT) and non-SOT patients at initial time of hospital visit with flu symptoms from potential H1N1 infection. Expression profiles were assessed using Ziplex® inflammation Xpress Chip containing 156 immune response gene probes. Clinical outcomes were assigned a binary score of good (no ICU) or poor (ICU stay or worse). Associations of markers to outcome were assessed by logistic regression analysis of all combinations of 1 to 3 markers to select approximately 50,000 promising combinations for computational validation. These underwent 10 rounds of 10-fold cross validation to determine mean AUCs.

### Sample Size and Categorization

A total of 32 samples taken on arrival to hospital were used for the analysis. These were split into 2 groups based on outcome: “good” (admitted to ward but no further complications) and “poor” (admission to ICU, required ventilator, required extracorporeal membrane oxygenation (ECMO), and/or death). 25 samples were classified in the “good” group and 7 samples in the “poor” group. To maximize discovery of predictive markers to assist clinicians in determining if a patient will have a high probability of more severe illness, the “good” group was restricted to ward admitted patients. An additional 7 patients that were not admitted to ward were excluded as these could be clearly diagnosed without the aid of additional biomarkers. In general, the selected models, when tested against the 7 excluded, non-ward patients, correctly predict these patients as having a low probability of poor outcome, consistent with the expected output of the models.

### Markers

162 markers were analyzed in total, including 6 clinical markers: tx (transplant recipient), sot (solid organ transplant), h1n1 (confirmed H1N1 infection), viral (confirmed viral infection), bacterial (confirmed bacterial infection), fungal (confirmed fungal infection), and 156 gene transcript markers from the Ziplex® Xpress Chip array.

### Statistical and Computational Analysis

For multivariate analysis all possible combinations of up to 3 markers were analyzed. There are 708,723 possible combinations of 3 or fewer markers in the set of 162 markers. To reduce computational complexity, a subset of combinations was selected that were likely to perform well by determining receiver operating characteristic (ROC) area under the curve (AUC) values from logistic regression using all data points. Since these AUC values are determined by testing on the same training set they are highly optimistic. Approximately 50,000 combinations were selected with the highest AUC values, also including all 162 combinations with only 1 marker, for additional analysis.

To better estimate the true predictive performance of each pre-selected combination, a 10-fold, balanced cross validation with 10 repeats was performed. Briefly, all samples were randomly partitioned into 10 folds (sub-groups) for which each fold was used as a test set for training against the other 9 folds. Folds were generated using striation so that the approximate ratio between groups was maintained in each fold. Because there was a much larger “good” outcome group than “poor” outcome group, for each training set a random selection of “good” samples, of equal size to the number of “poor” samples in that training set, was subsampled from all available “good” samples in that training set. This insured a balanced set of “good” and “bad” samples for each training set. Logistic regression was the model used and after log odds were determined for all 10 test sets (folds) the AUC was determined. This process was repeated 10 times for each combination generating 10 AUC values from which descriptive and differential statistics could further be performed. The random number generator was seeded with the same value for the cross-validation of each combination, ensuring the same samples were selected across all combinations, thus allowing paired comparisons to be made as appropriate.

### Results

### Clinical Markers

The six clinical markers were not selected among the top performers alone or in combination. This suggests the examined clinical markers are not strong predictors of outcome, or may in some cases have insufficient accuracy or sensitivity.

### Univariate Analysis

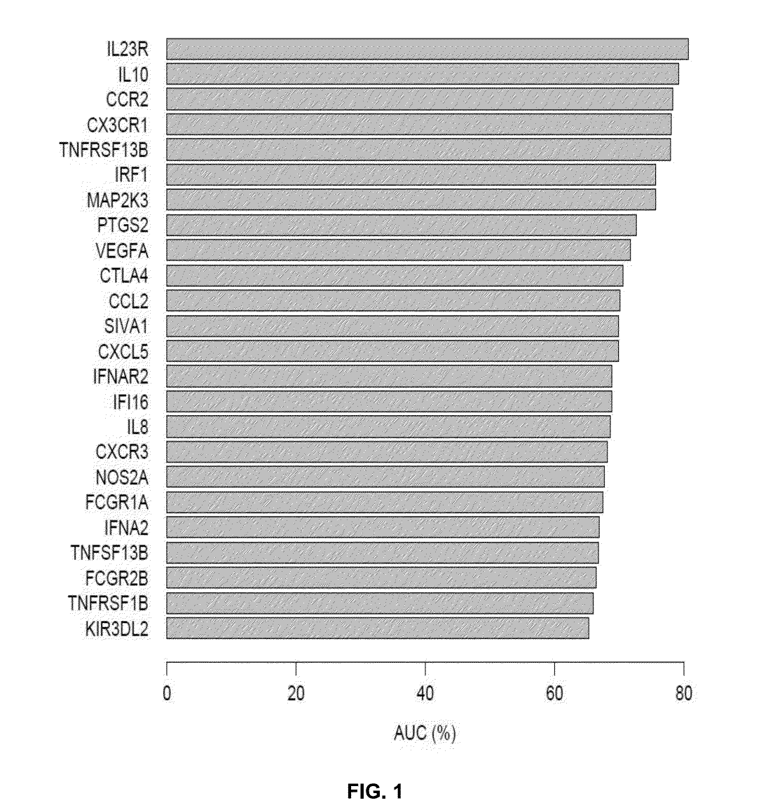

Results for all single markers are summarized in Table 1 and FIG. 1. The top 7 markers by mean AUC exhibited a mean AUC>0.75. The top markers show a drop-off of mean AUC after the top 7 that suggests a natural cut-off point for selection of important individual markers (Table 1 and FIG. 1).

Furthermore, 2-way ANOVA with repeated measures indicated that the top 7 markers do not have statistically significant differences in mean AUC with their AUC distributions taken into account. Once the 8th top marker is included, the result becomes significant (p<0.05) indicating this marker has a distinct distribution of AUCs compared to the top 7.

TABLE 1
Univariate analysis for all single markers

| | | | | | Relative |
|---|---|---|---|---|---|
| | | | Logistic Regression | | Expression |
| Markers | AUC | | Coefficients | | (Poor vs. Good) |
| marker1 | mean | SD | Intercept | marker1 | marker1 |
| IL23R | 0.806 | 0.0385 | 3.330 | −2.106 | Lower |
| IL10 | 0.792 | 0.0197 | −7.870 | 1.783 | Higher |
| TNFRSF13B | 0.783 | 0.0339 | 10.495 | −1.380 | Lower |
| CX3CR1 | 0.780 | 0.0555 | 8.321 | −1.228 | Lower |
| CCR2 | 0.779 | 0.0401 | −19.962 | 2.638 | Higher |
| MAP2K3 | 0.757 | 0.0351 | 15.485 | −1.909 | Lower |
| IRF1 | 0.756 | 0.0514 | 14.661 | −1.741 | Lower |
| PTGS2 | 0.727 | 0.0647 | 3.580 | −0.874 | Lower |
| VEGFA | 0.717 | 0.0565 | −9.037 | 1.674 | Higher |
| SIVA1 | 0.706 | 0.0375 | 6.097 | −1.101 | Lower |
| CCL2 | 0.701 | 0.0269 | 0.903 | −1.048 | Lower |
| IFI16 | 0.699 | 0.0522 | 9.060 | −1.405 | Lower |
| CTLA4 | 0.698 | 0.0620 | 3.775 | −1.214 | Lower |
| CXCL5 | 0.689 | 0.0515 | 7.156 | −1.772 | Lower |
| IL8 | 0.688 | 0.0340 | 6.786 | −1.068 | Lower |
| IFNAR2 | 0.686 | 0.0388 | 5.812 | −1.365 | Lower |
| NOS2A | 0.681 | 0.0274 | 0.497 | −1.010 | Lower |
| FCGR1A | 0.676 | 0.0478 | 1.537 | −0.668 | Lower |
| CXCR3 | 0.674 | 0.0482 | 2.114 | −1.201 | Lower |
| IFNA2 | 0.669 | 0.0429 | 0.104 | −0.664 | Lower |
| TNFRSF1B | 0.668 | 0.0486 | 7.920 | −1.419 | Lower |
| FCGR2B | 0.664 | 0.0589 | 6.584 | −1.077 | Lower |
| TNFSF13B | 0.660 | 0.0462 | 4.158 | −0.622 | Lower |
| h1n1 | 0.653 | 0.0439 | −2.251 | 2.069 | Higher |
| PIM1 | 0.646 | 0.0435 | 6.508 | −0.858 | Lower |
| HLA-DRA | 0.643 | 0.0408 | 6.117 | −0.840 | Lower |
| KIR3DL2 | 0.641 | 0.0553 | 6.487 | −0.806 | Lower |
| MIF | 0.637 | 0.0627 | 5.353 | −0.807 | Lower |
| IL6R | 0.630 | 0.0532 | 3.133 | −1.123 | Lower |
| ADAM17 | 0.630 | 0.0355 | −14.143 | 2.091 | Higher |
| PLAU | 0.625 | 0.0472 | 5.003 | −0.967 | Lower |
| C1QA | 0.618 | 0.0727 | 1.315 | −0.500 | Lower |
| CD14 | 0.614 | 0.0788 | −5.990 | 0.639 | Higher |
| CD22 | 0.613 | 0.0572 | 1.401 | −0.849 | Lower |
| FCGR2A | 0.610 | 0.0630 | 3.550 | −0.524 | Lower |
| CCR9 | 0.607 | 0.0545 | 3.860 | −0.650 | Lower |
| F3 | 0.602 | 0.0547 | 0.417 | −0.790 | Lower |
| CXCL10 | 0.602 | 0.0409 | 0.727 | −0.646 | Lower |
| HAVCR2 | 0.597 | 0.0717 | −6.657 | 1.113 | Higher |
| IRAK3 | 0.596 | 0.0761 | −5.238 | 0.657 | Higher |
| MMP9 | 0.595 | 0.0945 | −4.224 | 0.453 | Higher |
| CSF3 | 0.589 | 0.0255 | 0.678 | −0.586 | Lower |
| MPO | 0.589 | 0.0477 | 2.655 | −0.600 | Lower |
| CXCR4 | 0.589 | 0.0847 | −10.444 | 1.051 | Higher |
| IL18 | 0.589 | 0.0604 | −5.940 | 0.987 | Higher |
| IRF8 | 0.584 | 0.0852 | −4.462 | 0.414 | Higher |
| CSF2 | 0.583 | 0.0771 | −0.282 | −0.518 | Lower |
| IL5 | 0.579 | 0.0728 | 1.658 | −0.629 | Lower |
| IFNG | 0.578 | 0.0981 | −5.640 | 1.103 | Higher |
| IL13 | 0.576 | 0.0833 | 1.108 | −0.606 | Lower |
| IRF5 | 0.576 | 0.0637 | −3.619 | 0.556 | Higher |
| IL1B | 0.575 | 0.0511 | 1.488 | −0.396 | Lower |
| S100A12 | 0.573 | 0.0662 | −5.113 | 0.345 | Higher |
| SYVN1 | 0.570 | 0.0893 | −7.437 | 0.924 | Higher |
| TIMP1 | 0.565 | 0.0463 | 2.775 | −0.423 | Lower |
| IL17RA | 0.564 | 0.0847 | −6.133 | 0.733 | Higher |
| VCAM1 | 0.561 | 0.0933 | −1.139 | −0.151 | Lower |
| MS4A1 | 0.561 | 0.0711 | −2.574 | 0.309 | Higher |
| IFNGR1 | 0.561 | 0.0769 | −7.136 | 0.771 | Higher |
| TGFB2 | 0.560 | 0.0543 | −0.924 | −0.430 | Lower |
| SERPINA1 | 0.560 | 0.0531 | −1.064 | −0.058 | Lower |
| CD40LG | 0.554 | 0.0847 | 2.298 | −0.358 | Lower |
| IL18BP | 0.553 | 0.0567 | 1.981 | −0.608 | Lower |
| IL6 | 0.551 | 0.0389 | 1.472 | −0.603 | Lower |
| FOXP3 | 0.551 | 0.0642 | 0.678 | −0.553 | Lower |
| HAVCR1 | 0.550 | 0.0671 | 0.479 | −0.561 | Lower |
| MMP3 | 0.550 | 0.1033 | −1.680 | 0.551 | Higher |
| CD86 | 0.546 | 0.0452 | 2.953 | −0.936 | Lower |
| RAGE | 0.542 | 0.0782 | 1.888 | −0.485 | Lower |
| TYK2 | 0.540 | 0.0632 | 1.424 | −0.450 | Lower |
| bacterial | 0.536 | 0.0257 | −1.674 | 0.863 | Higher |
| FASLG | 0.535 | 0.0755 | 0.802 | −0.491 | Lower |
| IL2RA | 0.535 | 0.0890 | 1.726 | −0.512 | Lower |
| PTX3 | 0.532 | 0.0894 | −0.223 | −0.328 | Lower |
| IL7 | 0.530 | 0.1111 | −1.103 | −0.135 | Lower |
| TNFRSF11B | 0.529 | 0.0751 | 1.058 | −0.425 | Lower |
| CXCL2 | 0.528 | 0.0540 | 0.519 | −0.409 | Lower |
| ACADVL | 0.527 | 0.0451 | 4.755 | −0.889 | Lower |
| IKBKE | 0.525 | 0.0662 | 2.963 | −0.720 | Lower |
| CD40 | 0.521 | 0.0943 | −0.249 | −0.455 | Lower |
| CD8A | 0.521 | 0.0683 | −3.754 | 0.398 | Higher |
| TGFB3 | 0.521 | 0.0530 | −1.162 | −0.179 | Lower |
| CD58 | 0.519 | 0.1112 | −5.654 | 0.695 | Higher |
| SERPINE1 | 0.518 | 0.0600 | 0.410 | −0.372 | Lower |
| IL7R | 0.514 | 0.0860 | −2.673 | 0.313 | Higher |
| DHFR | 0.513 | 0.0923 | −3.930 | 0.505 | Higher |
| TLR4 | 0.510 | 0.1013 | −0.202 | −0.142 | Lower |
| viral | 0.509 | 0.0950 | −1.145 | −16.421 | Lower |
| CTSK | 0.506 | 0.0634 | −0.289 | −0.129 | Lower |
| C3 | 0.502 | 0.0706 | −0.235 | −0.244 | Lower |
| IL1A | 0.499 | 0.0670 | −0.812 | −0.204 | Lower |
| TNF | 0.495 | 0.1104 | 0.308 | −0.405 | Lower |
| SOCS3 | 0.493 | 0.0764 | −2.352 | 0.147 | Higher |
| HSPA1A | 0.488 | 0.0988 | −3.882 | 0.267 | Higher |
| KIR3DL1 | 0.487 | 0.0794 | −0.069 | −0.268 | Lower |
| LCN2 | 0.487 | 0.0832 | −2.265 | 0.190 | Higher |
| TLR2 | 0.484 | 0.1047 | −2.294 | 0.213 | Higher |
| CD19 | 0.482 | 0.0903 | −2.809 | 0.314 | Higher |
| RAGE | 0.478 | 0.0532 | −0.638 | −0.155 | Lower |
| CCR1 | 0.477 | 0.1019 | −1.811 | 0.061 | Higher |
| TNFSF11 | 0.476 | 0.0845 | −1.866 | 0.512 | Higher |
| IL18R1 | 0.474 | 0.1260 | −2.615 | 0.305 | Higher |
| IL12B | 0.472 | 0.0695 | −1.121 | −0.075 | Lower |
| IL2 | 0.472 | 0.0934 | −1.787 | 0.638 | Higher |
| RAGE | 0.470 | 0.0699 | −1.010 | −0.139 | Lower |
| CCL5 | 0.467 | 0.1175 | 1.311 | −0.332 | Lower |
| PTGES2 | 0.466 | 0.0681 | −2.421 | 0.539 | Higher |
| fungal | 0.465 | 0.0823 | −1.232 | −15.334 | Lower |
| IL15 | 0.465 | 0.0967 | −3.547 | 0.641 | Higher |
| CSF1 | 0.462 | 0.1105 | −1.629 | 0.207 | Higher |
| ICAM1 | 0.462 | 0.0996 | 1.171 | −0.464 | Lower |
| ITGAL | 0.455 | 0.0556 | 2.056 | −0.667 | Lower |
| MSH2 | 0.454 | 0.1139 | −1.753 | 0.098 | Higher |
| EGR1 | 0.451 | 0.0910 | −1.378 | 0.031 | Higher |
| IL1RN | 0.449 | 0.0929 | −1.324 | 0.007 | Higher |
| SELE | 0.448 | 0.0922 | −1.249 | −0.007 | Lower |
| EBI3 | 0.442 | 0.0896 | −2.279 | 0.125 | Higher |
| CD3Z | 0.441 | 0.1834 | −0.989 | −0.043 | Lower |
| CD69 | 0.439 | 0.0800 | −2.212 | 0.200 | Higher |
| IL12RB2 | 0.439 | 0.0709 | −1.233 | −0.013 | Lower |
| IL12A | 0.438 | 0.1002 | −4.343 | 1.065 | Higher |
| KIR3DS1 | 0.437 | 0.0875 | −0.760 | −0.188 | Lower |
| CCR5 | 0.435 | 0.1579 | −2.806 | 0.269 | Higher |
| LTA | 0.433 | 0.1181 | −0.381 | −0.075 | Lower |
| sot | 0.432 | 0.1449 | −1.435 | 0.742 | Higher |
| CRP | 0.427 | 0.0972 | −0.778 | −0.146 | Lower |
| IL4 | 0.425 | 0.0846 | −2.261 | 0.124 | Higher |
| HMOX1 | 0.423 | 0.1105 | −1.734 | 0.075 | Higher |
| IFNB1 | 0.423 | 0.1045 | −1.390 | 0.123 | Higher |
| MX1 | 0.419 | 0.0853 | −0.383 | −0.142 | Lower |
| MCL1 | 0.418 | 0.0941 | 2.290 | −0.331 | Lower |
| PLA2G7 | 0.416 | 0.0934 | −1.585 | 0.069 | Higher |
| ADORA3 | 0.414 | 0.1013 | −2.947 | 0.367 | Higher |
| CXCR6 | 0.411 | 0.0820 | −2.427 | 0.412 | Higher |
| PTPRC | 0.411 | 0.1337 | −1.005 | −0.054 | Lower |
| CXCL1 | 0.410 | 0.0695 | −3.559 | 0.720 | Higher |
| IL17A | 0.408 | 0.0900 | −1.781 | 0.382 | Higher |
| CD80 | 0.407 | 0.1032 | −1.776 | 0.099 | Higher |
| IRF7 | 0.403 | 0.1141 | −1.645 | 0.056 | Higher |
| ITGB2 | 0.401 | 0.1122 | 1.715 | −0.289 | Lower |
| CD1D | 0.400 | 0.0585 | −1.892 | 0.329 | Higher |
| TGFBR2 | 0.399 | 0.0729 | −0.858 | −0.067 | Lower |
| SOCS1 | 0.397 | 0.1011 | −1.406 | 0.023 | Higher |
| IL8RB | 0.395 | 0.0724 | −0.584 | −0.071 | Lower |
| MMP13 | 0.395 | 0.0974 | −1.218 | 0.094 | Higher |
| CD28 | 0.393 | 0.0934 | −1.530 | 0.077 | Higher |
| IL1R1 | 0.391 | 0.0815 | −2.472 | 0.173 | Higher |
| TGFB3 | 0.386 | 0.1594 | −1.390 | 0.208 | Higher |
| IL23A | 0.383 | 0.1212 | −1.106 | −0.057 | Lower |
| CYBB | 0.381 | 0.0951 | 1.518 | −0.297 | Lower |
| TGFB3 | 0.374 | 0.1044 | −1.476 | 0.133 | Higher |
| CD70 | 0.371 | 0.0990 | −3.904 | 0.543 | Higher |
| ELA2 | 0.371 | 0.1083 | −1.826 | 0.090 | Higher |
| SERPING1 | 0.369 | 0.0958 | −0.602 | −0.102 | Lower |
| MMP1 | 0.368 | 0.1302 | −2.280 | 0.415 | Higher |
| tx | 0.366 | 0.1513 | −1.386 | 0.470 | Higher |
| TNFSF14 | 0.359 | 0.0957 | −1.669 | 0.082 | Higher |
| CD27 | 0.351 | 0.1290 | −2.675 | 0.275 | Higher |
| CD4 | 0.346 | 0.0975 | −1.139 | −0.032 | Lower |
| LTB | 0.340 | 0.1062 | −3.477 | 0.319 | Higher |
| TGFB1 | 0.328 | 0.1094 | −1.616 | 0.130 | Higher |
| PTGES | 0.328 | 0.0916 | −1.165 | −0.073 | Lower |

The top 7 markers were: IL23R, IL10, TNFRSF13B, CX3CR1, CCR2, MAP2K3 and IRF1. These markers have mean AUC values between 0.75 and 0.81 as shown in Table 2:

TABLE 2
Top 7 single markers with highest AUC values

| | mean | Relative Expression |
|---|---|---|
| marker | AUC | (Poor vs. Good) |
| IL23R | 0.806 | Lower |
| IL10 | 0.792 | Higher |
| TNFRSF13B | 0.783 | Lower |
| CX3CR1 | 0.780 | Lower |
| CCR2 | 0.779 | Higher |
| MAP2K3 | 0.757 | Lower |
| IRF1 | 0.756 | Lower |

### Multivariate Analysis

To select for combinations of markers that improve on the performance of the individual markers, those markers with a larger AUC, especially those with mean AUC>0.90, were examined. There were 107 combinations with mean AUC>0.90, of which 1 was a 2-marker combination and the other 106 were 3-marker combinations.

The top 2-marker combination (mean AUC=0.90) was a combination of the 2 top single markers (IL23R and IL10). This combination has a significantly different AUC distribution than the next highest 2-marker combination. However, the following 18 2-marker combinations did not differ significantly from each other (2-way ANOVA with repeated measures) and each exhibited mean AUC values between 0.82 and 0.88. The top 19 2-marker combinations are shown in Table 3:

TABLE 3
Top 19 2-marker combinations with the highest mean AUC

| | | | | Logistic Regression | | | Relative Expression | |
|---|---|---|---|---|---|---|---|---|
| Markers | | AUC | | Coefficients | | | (Poor vs. Good) | |
| marker1 | marker2 | Mean | SD | Intercept | marker1 | marker2 | marker1 | marker2 |
| IL10 | IL23R | 0.904 | 0.0150 | −4.725 | 2.830 | −3.248 | Higher | Lower |
| FCGR1A | IL10 | 0.877 | 0.0328 | −4.924 | −1.071 | 2.229 | Lower | Higher |
| IL10 | MAP2K3 | 0.869 | 0.0504 | 8.153 | 1.871 | −1.855 | Higher | Lower |
| CCR2 | IFI16 | 0.865 | 0.0551 | 91.540 | 27.053 | −38.474 | Higher | Lower |
| IFNG | PTGS2 | 0.864 | 0.0486 | −1.292 | 1.749 | −1.222 | Higher | Lower |
| CD14 | IL23R | 0.860 | 0.0405 | −14.764 | 3.208 | −4.664 | Higher | Lower |
| CCR2 | FCGR1A | 0.860 | 0.0517 | −17.463 | 3.647 | −2.327 | Higher | Lower |
| IL23R | TLR2 | 0.858 | 0.0559 | −10.547 | −5.917 | 4.631 | Lower | Higher |
| CX3CR1 | MMP13 | 0.847 | 0.0383 | 15.052 | −2.030 | 0.789 | Lower | Higher |
| IL2 | MAP2K3 | 0.843 | 0.0546 | 78.565 | 7.133 | −9.867 | Higher | Lower |
| CTLA4 | IL23R | 0.838 | 0.0525 | 19.279 | −2.801 | −4.135 | Lower | Lower |
| IL2 | IL23R | 0.834 | 0.0596 | 8.987 | 2.429 | −5.458 | Higher | Lower |
| IFNB1 | TNFRSF13B | 0.832 | 0.0212 | 19.577 | 2.217 | −2.723 | Higher | Lower |
| IL15 | IL23R | 0.829 | 0.0371 | −0.383 | 1.734 | −3.213 | Higher | Lower |
| FCGR2B | IL10 | 0.826 | 0.0559 | −0.212 | −1.266 | 2.205 | Lower | Higher |
| MMP3 | RAGE | 0.825 | 0.0460 | 43.798 | 4.086 | −7.594 | Higher | Lower |
| CD1D | MIF | 0.823 | 0.0587 | 17.008 | 2.117 | −2.708 | Higher | Lower |
| MMP3 | SIVA1 | 0.822 | 0.0417 | 19.665 | 2.287 | −3.375 | Higher | Lower |
| ADORA3 | TNFRSF13B | 0.819 | 0.0665 | 0.910 | 4.814 | −2.799 | Higher | Lower |

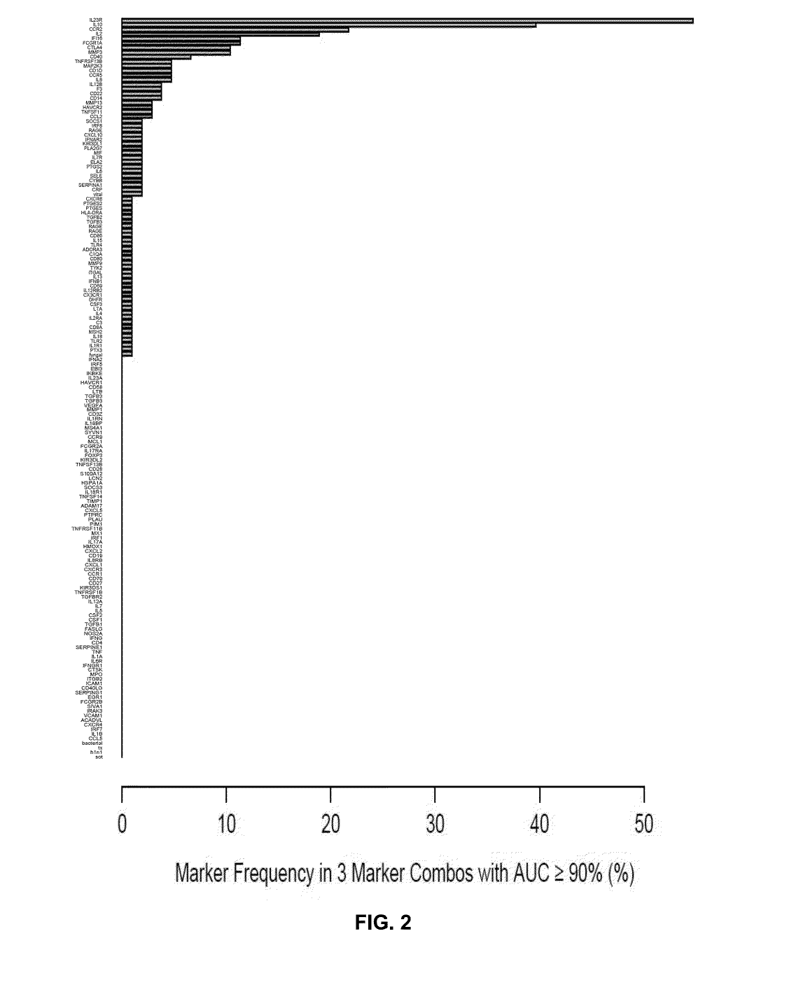

Among the 106 3-marker combinations with a mean AUC>0.90, FIG. 2 shows the frequency with which each marker is found among these combinations. There are 9 markers that show up in >5% of these 106 combinations. These are, in order of frequency: IL23R, IL10, CCR2, IL2, IF116, FCGR1A, CTLA4, MMP3, CD40. The top 3 are among the top 7 top performing individual markers. The remaining markers do not perform as well individually, but are frequently found in high performing combinations and are thus likely to play a synergistic role in improving performance.

The top 15 3-marker models do not differ significantly from each other (2-way ANOVA with repeated measures). These combinations are listed in Table 4:

TABLE 4
Top 15 3-marker combinations with the highest mean AUC. “H” = higher; “L” = lower

| | | | | | Logistic Regression | | | | Relative Expression | | |
|---|---|---|---|---|---|---|---|---|---|---|---|
| Markers | | | AUC | | Coefficients | | | | (Poor vs. Good) | | |
| M1 | M2 | M3 | mean | SD | Intercept | M1 | M2 | M3 | M1 | M2 | M3 |
| IL15 | IL2 | IL23R | 0.972 | 0.0167 | −4.847 | 82.776 | 99.306 | −184.127 | H | H | L |
| CXCR6 | IL2 | IL23R | 0.972 | 0.0227 | 181.228 | 56.993 | 59.122 | −184.529 | H | H | L |
| IL2 | IL23R | IRF8 | 0.969 | 0.0290 | −3682.173 | 415.587 | −837.336 | 648.204 | H | L | H |
| IL10 | IL2 | IL23R | 0.959 | 0.0181 | −290.982 | 297.656 | 283.633 | −530.813 | H | H | L |
| CD69 | IL2 | IL23R | 0.955 | 0.0318 | −639.424 | 458.512 | 822.921 | −1156.990 | H | H | L |
| IL10 | IL23R | MMP3 | 0.950 | 0.0223 | −9.151 | 3.870 | −3.640 | 1.641 | H | L | H |
| CCL2 | IL10 | TNFSF11 | 0.948 | 0.0412 | −848.024 | −157.985 | 253.892 | 101.911 | L | H | H |
| CD14 | IL2 | IL23R | 0.947 | 0.0213 | −8515.762 | 1734.018 | 1420.840 | −2688.147 | H | H | L |
| IL2 | IL23R | PTGES | 0.947 | 0.0179 | 646.474 | 91.135 | −405.404 | 96.884 | H | L | H |
| C3 | CTLA4 | IL23R | 0.946 | 0.0267 | 1783.022 | 684.168 | −684.852 | −848.737 | H | L | L |
| IL2 | IL23R | KIR3DL1 | 0.944 | 0.0377 | −15.608 | 124.333 | −357.180 | 149.721 | H | L | H |
| CD8A | IL2 | IL23R | 0.944 | 0.0386 | −123.950 | 58.587 | 104.922 | −160.758 | H | H | L |
| CCR5 | IL2 | IL8 | 0.939 | 0.0357 | 2292.431 | 375.998 | 292.513 | −636.015 | H | H | L |
| CXCL10 | IL2 | IL23R | 0.937 | 0.0368 | 319.028 | 183.399 | 323.321 | −576.817 | H | H | L |
| IL10 | IL23R | IL7R | 0.937 | 0.0224 | −14088.605 | 2824.883 | −1035.239 | 1157.063 | H | L | H |

Of the 15 3-marker combinations listed in Table 4, IL23R appears 13 times and the pair of markers IL23R and IL2 occur together in 10 of the top 15 combinations. This pair of markers, without a third, has a mean AUC of 0.83 (and occurs within the group of 18 statistically similar 2-marker models described above (see Table 3). Welch's t-test comparing mean AUC values between these 2 groups (18 2-marker vs. 15 3-marker) shows a highly significant difference (p<<<0.05) suggesting that the addition of the third marker does indeed significantly improve the predictive performance of already well performing pairs of markers.

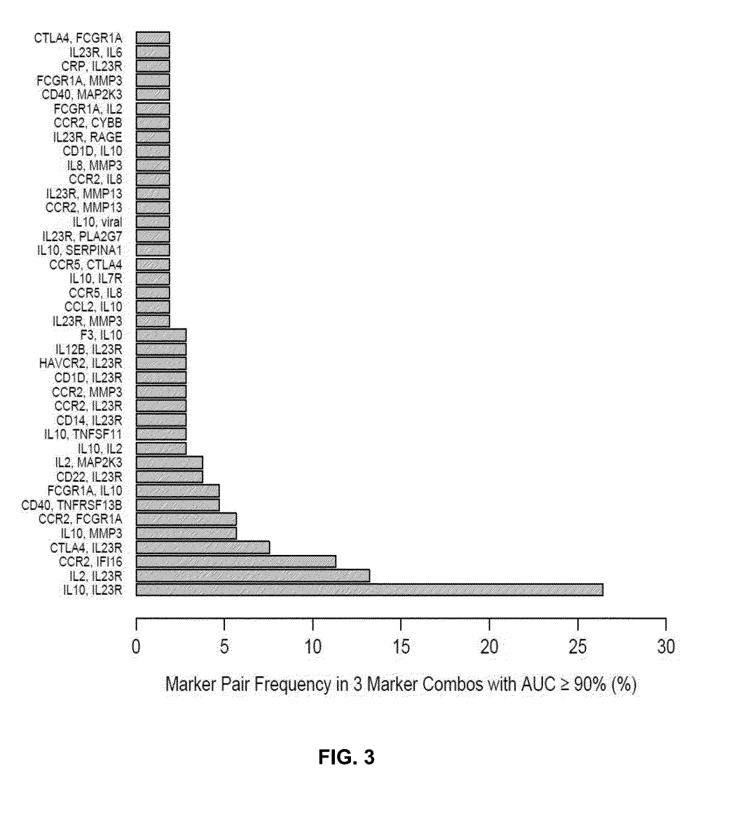

FIG. 3 shows the frequency distribution of marker pairs that occur in the 106 3-marker combinations with AUC>0.90. The 6 pairs that occur in >5% of combinations are listed in Table 5:

TABLE 5
Top 6 marker pairs from 3-marker combinations with AUC > 0.90

| Marker Pair | % |
|---|---|
| IL10, IL23R | 26.4 |
| IL2, IL23R | 13.2 |
| CCR2, IFI16 | 11.3 |
| CTLA4, IL23R | 7.5 |
| IL10, MMP3 | 5.7 |
| CCR2, FCGR1A | 5.7 |

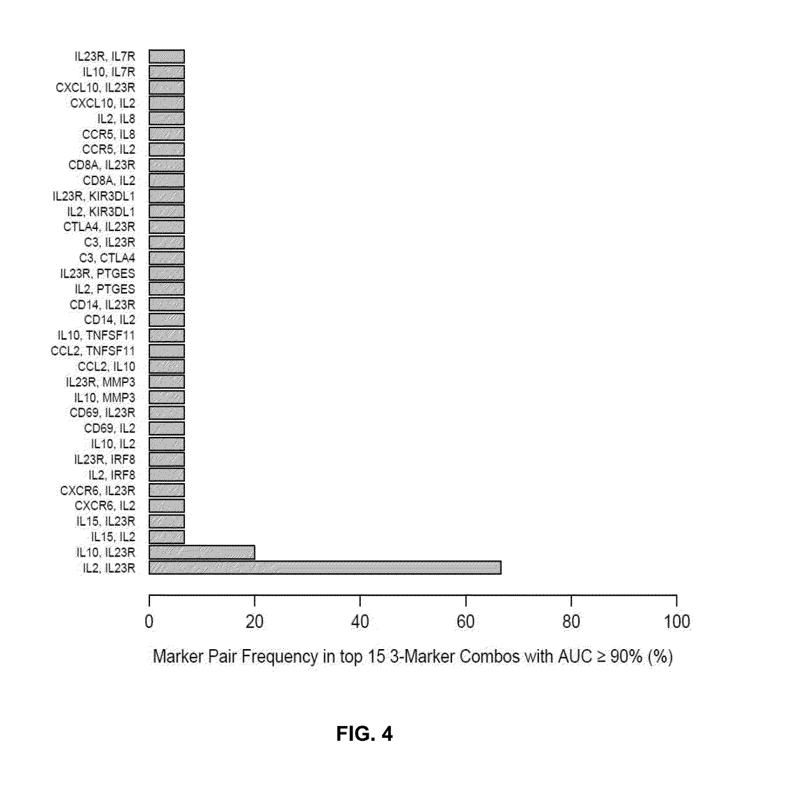

FIG. 4 shows the distribution of marker pairs in the top 15 3-marker combinations with an AUC greater than or equal to 90%. When looking only at the top 15 3-marker combinations shown in FIG. 4, there are only 2 pairs which occur in more than one combination: IL2 and IL23R that have a marker pair frequency of 67% and IL10 and IL23R that have a marker pair frequency of 20%.

### SUMMARY

The biomarkers or combinations of biomarkers that are likely to most predictive of subjects likely to progress to severe illness from an infection with H1N1 influenza virus include:
1. The individual markers: IL23R, IL10, TNFRSF13B, CX3CR1, CCR2, MAP2K3 and IRF1.
2. The pair of IL23R+IL2, especially when combined with a third marker, particularly: IL15, CXCR6, IRF8, IL10, CD69, CD14, PTGES, KIR3DL1, CD8A or CXCL10.
3. The pair of IL23R+IL10, especially when combined with a third marker, particularly: IL2, MMP3 or IL7R.

The direction in which each marker differs in the “poor” group compared to the “good” (i.e. higher=“poor”>“good”; lower=“poor”<“good”) is indicated in Tables above. In the Tables 1, 3 and 4 the coefficients of the logistic regression model, using all 32 samples, is indicated. From these models optimum log odds thresholds may be determined for each model. However, the models should hold across different assays. For clinical use, a more quantitative assay may be used and assay-specific coefficients and thresholds may be determined for the markers and marker combinations described herein.

While the present disclosure has been described with reference to what are presently considered to be the preferred examples, it is to be understood that the disclosure is not limited to the disclosed examples. To the contrary, the disclosure is intended to cover various modifications and equivalent arrangements included within the spirit and scope of the appended claims.

All publications, patents and patent applications are herein incorporated by reference in their entirety to the same extent as if each individual publication, patent or patent application was specifically and individually indicated to be incorporated by reference in its entirety.

1. A method of identifying a subject having, or at risk of developing, severe illness from infection with an influenza virus, the method comprising:
(a) determining a level of one or more biomarkers listed in Table 1 in a test sample from the subject; and
(b) comparing the level of the one or more biomarkers in the test sample to a level of the one or more biomarkers in a control sample, wherein a difference or similarity in the level of the one or more biomarkers in the test sample compared to the control sample indicates that the subject has, or is at risk of developing, severe illness from infection with the influenza virus.

2. The method of claim 1, wherein the influenza virus is H1N1 (swine flu).

3. The method of claim 1, wherein the one or more biomarkers includes a biomarker selected from IL23R, IL10, TNFRSF13B, CX3CR1, CCR2, MAP2K3 and IRF1.

4. The method of claim 1, wherein the control sample is representative of the level of the one or more biomarkers in subjects with an influenza infection who do not develop severe illness.

5. The method of claim 4, wherein the marker is IL23R and a lower level of IL23R in the test sample relative to the control sample is indicative of the subject having, or being at risk of developing, severe illness.

6. The method of claim 4, wherein the marker is IL10 and a higher level of IL10 in the test sample relative to the control sample is indicative of the subject having, or being at risk of developing, severe illness.

7. The method of claim 4, wherein the biomarker marker is TNFRS13B, CX3R1, MAP2K3 or IRF1 and a lower level of TNFRS13B, CX3R1, MAP2K3 or IRF1 in the test sample relative to the control sample is indicative of the subject having, or being at risk of developing, severe illness.

8. The method of claim 4, wherein the biomarker is CCR2 and a higher level of CCR2 in the test sample relative to the control sample is indicative of the subject having, or being at risk of developing, severe illness.

9. The method of claim 1, wherein step a) comprises determining the level of two or more biomarkers listed in Table 3.

10. The method of claim 9, wherein the two or more biomarkers include IL23R and IL2.

11. The method of claim 10, further comprising testing the sample for a biomarker selected from the group consisting of IL15, CXCR6, IRF8, IL10, CD69, CD14, PTGES, KIR3DL1, CD8A and CXCL10.

12. The method of claim 9, wherein the two or more biomarkers include IL23R and IL10.

13. The method of claim 12, further comprising testing the sample for a biomarker selected from the group consisting of IL2, MMP3 and IL7R.

14. The method of claim 1, wherein step a) comprises determining the level of three or more biomarkers listed in Table 4.

15. The method of claim 1, wherein step a) comprises determining the level of two or more biomarkers and step b) comprises multivariate analysis.

16. The method of claim 1, wherein the control sample is a sample from the subject at an earlier time point.

17. The method of claim 1, wherein the test sample is a blood sample, serum sample or plasma sample.

18. The method of claim 1, wherein determining the level of one or more biomarkers comprises testing the sample for a nucleic acid molecule or polypeptide encoding for all or part of the biomarker.

19. The method of claim 1, further comprising treating a subject identified as having, or at risk of developing, severe illness from infection with an influenza virus for severe illness.

20. A kit for determining whether a subject has, or is at risk of developing, severe illness from infection with an influenza virus, the kit comprising two or more binding agents selective for two or more of the biomarkers listed in Table 1.
